(19) United States
(12) Patent Application Publication
(10) Pub. No.: US 2017/0202502 A1
(43) Pub. Date: Jul. 20, 2017

(54) SENSOR AND SYSTEM FOR INCONTINENCE EVENT DETECTION AND MEDICAL PATIENT MONITORING

(71) Applicant: FIT Assist Medical Inc., Vancouver (CA)

(72) Inventor: Eugen Cretu-Petra, Dandenong (AU)

(21) Appl. No.: 15/407,549

(22) Filed: Jan. 17, 2017

Related U.S. Application Data
(60) Provisional application No. 62/279,112, filed on Jan. 15, 2016.

(51) Int. Cl. A61B 5/20; G01K 7/22; G01N 27/22; A61B 5/00

(52) U.S. Cl. CPC A61B 5/208; A61B 5/6804; A61B 5/0022; G01K 7/22; G01N 27/223

(57) ABSTRACT

A sensor for detecting and monitoring incontinence events using reactive and resistive elements comprising a reusable sensor for attachment to undergarments, with capabilities for measurement of relative and absolute quantities of urine released by the patient, and for detection of defecation events. The sensor also incorporates automated AC impedance measurement circuitry and remote monitoring and alerting capabilities.

### CROSS-REFERENCE TO RELATED APPLICATION

The present application claims the benefits, under 35 U.S.C.§119(e), of U.S. Provisional Application Ser. No. 62/279,112 filed Jan. 15, 2016 entitled “Sensor and System for Incontinence Event Detection and Medical Patient Monitoring” which is incorporated herein by this reference.

### TECHNICAL FIELD

The invention relates to monitoring systems for monitoring various functions of infants or patients in nursing homes, hospitals or home care, and in particular sensors for use in such systems.

### BACKGROUND OF THE INVENTION

An aging population and increased average human lifespans, coupled with improving and expanding healthcare and elderly care facilities, have led to growing interest and demand for technologies and systems for sensing and monitoring incontinence events such as involuntary urination and defecation, and for automatically alerting medical staff and other caregivers as to changes in the condition of patients who may be prone to incontinence events. Such technologies and systems are also of interest to parents caring for infants, and in general for the care of patients of any age with incontinence-related disorders.

Various systems are known for monitoring the diapers of infants or incontinent patients in health care facilities to detect urination or defecation. U.S. Pat. No. 5,903,222 to Kawarizadeh et al. discloses a detector for detecting wetness conditions in diapers using a capacitive sensor in a housing attached to the exterior surface of the garment being monitored. If a wetness condition is detected a signal is transmitted to a central monitoring station. That sensor lacks the capability for progressive monitoring of urinary output and is susceptible to stray body capacitances. U.S. Pat. No. 6,570,053 to Roe et al. discloses a diaper which has an electrical sensor to detect signals that correlate to an impending elimination of bodily waste. U.S. Pat. No. 7,977,529 to Bergman et al. discloses an incontinence management system for monitoring wetness events in the diapers of multiple patients. The sensors in such a system are located within the diaper so the diapers in such system are designed for use for only a few days as the sensors only last for one incontinent event.

Recently, in US patent application publication no. 2014/0266736, issuing to the Applicant herein on 17 Jan. 2017 as U.S. Pat. No. 9,545,342, a system for monitoring urination and defecation events using a plurality temperature sensors has been described. Said system is capable of detection and monitoring of such events, but cannot readily measure relative or absolute quantity of discharged amounts of urine or other substances as a function of time. The present sensor described below addresses these drawbacks and others and is suitable for use in the system disclosed in US patent application publication no. 2014/0266736, which is incorporated herein by reference.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

There is provided a sensing apparatus for use in the detection and assessment of urination events in a garment worn by a person, said sensing apparatus comprising a plurality of capacitive elements, each said capacitive element comprising a planar conductor having a first planar surface for releasable attachment to the outer surface of said garment and an opposed surface mounted on and dielectrically spaced from a grounded conductive shielding layer common to said plurality of capacitive elements, the planar conductor of each said capacitive element being isolatedly spaced from each adjacent planar conductor whereby each said planar conductor in combination with said grounded shielding layer forms a first plurality of capacitive elements and said outer surface of said garment in combination with a pair of adjacent planar conductors forms a second capacitive element in which a portion of said outer surface of said garment connecting said adjacent planar conductors forms the dielectric element of said second capacitive element.

More particularly a sensing apparatus for the detection and assessment of urination events in a garment worn by a person is provided, said sensing apparatus comprising a plurality of capacitive elements, each said capacitive element comprising a planar conductor having a first planar surface for releasable attachment to the outer surface of said garment and an opposed surface mounted on and dielectrically spaced from a grounded conductive shielding layer common to said plurality of capacitive elements, the planar conductor of each said capacitive element being isolatedly spaced from each adjacent planar conductor whereby said outer surface of said garment in combination with a pair of adjacent planar conductors forms a capacitive element.

There is further provided a system for using capacitance measuring techniques to monitor relative quantities of urinary output of a person wearing a garment as a function of time, the system comprising the afore-mentioned sensing apparatus, a function generator for generating an AC signal of selected frequency communicated to the sensing apparatus, and a selective amplifier for generating from an AC signal from said sensing apparatus a signal representing a variable voltage for processing in an analog-to-digital processor to provide a relative indication of urinary output in said garment. A method of using such a system for the monitoring of absolute quantities of urinary output in a person's garment is also disclosed.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a schematic representation of the sensor apparatus in plan view.

FIG. 2 is a cross-section view illustrating the connection of the sensor to a diaper surface.

FIG. 3 is a transverse cross-section of the sensor taken along lines 3-3 in FIG. 1, when attached to a diaper as shown in FIG. 2.

FIG. 4 is a schematic circuit diagram representation of the sensor apparatus.

FIG. 5 is a functional schematic representation of sensor output analysis circuitry with sensor element representation.

FIG. 6 is a circuit diagram of an embodiment of the sensor output analysis circuitry.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

The following embodiment is described for detecting and monitoring incontinence events using reactive and resistive elements comprising a reusable sensor for attachment to undergarments, with capabilities for measurement of relative and absolute quantities of urine released by the patient, and for detection of defecation events. The invention also incorporates automated AC impedance measurement circuitry and remote monitoring and alerting capabilities.

The described embodiment includes a robust, flexible, reusable sensor apparatus for installation on the external surface of diapers or other undergarments that are to be worn by the patient. The sensor apparatus when so worn is capable of detecting both urination and defecation events, and has other capabilities. The sensor apparatus is designed to detect multiple urinations and multiple defecations in any diaper and also to sense each urination and provide an indication of the total quantity of liquid in the diaper at any desired time. The apparatus includes certain resistive and capacitive sensing elements. Also included are supporting electronic components for analysing the states of the sensing elements and for wirelessly or otherwise communicating results such as detected incontinence events to medical personnel or to other authorized parties. Further detail regarding the sensor apparatus and electronic components, and regarding the capabilities of the invention, is given in the following text and representative figures.

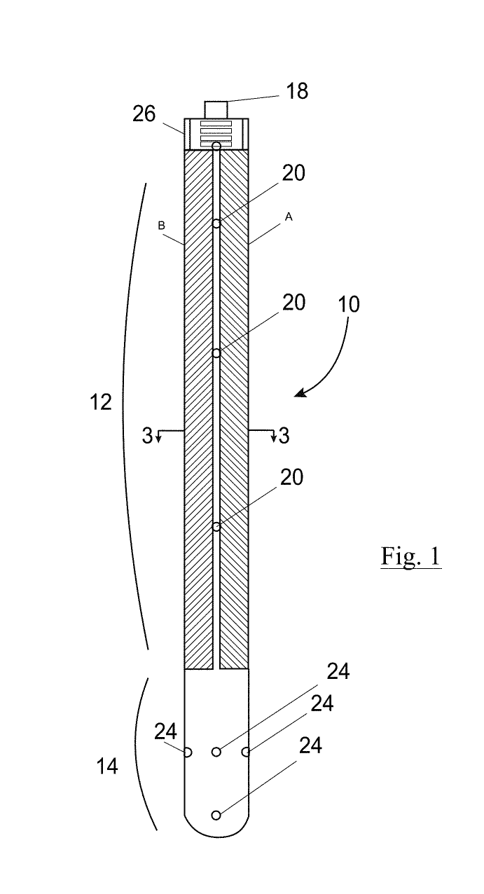

First, in regard to the sensor apparatus, FIG. 1 is a representation of the layout and relative location of the sensing elements on the sensor apparatus, designated by reference numeral 10. Sensor 10 comprises a flexible band of fixed width, plus a rigid component 26 at its end used as a handle and a multi-conductor connector 18 for the purpose of connection to external electronics. Representative values for the width of the flexible band are 10 mm to 50 mm, although other widths are possible within the scope of the present invention. The connector 18 may be of a commonly used type such as a micro USB or any other suitable type of connector. In some circumstances, encapsulating elements for sealing against liquid penetration may be utilized surrounding the connector and potential mating connectors.

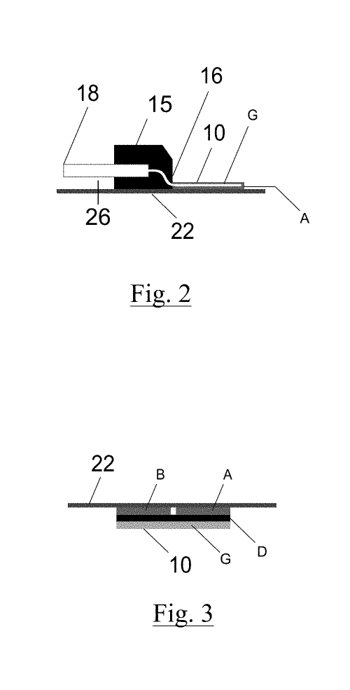

Sensor 10 has two functional areas for incontinence event detection, urination detection area 12 and defecation detection area 14 respectively. In one preferred embodiment, sensor 10 has as a substrate a flexible printed circuit board (PCB) 16 with no memory in regard to shape after being bent. Such flexibility enables re-use of sensor 10. The purpose of the handle 26 is to facilitate manipulation of the apparatus and guide it mechanically into an external connection to ensure an optimal connection. As shown in FIG. 2, handle 26 can be used to hold the sensor firmly in a receptacle 15 attached to the surface 22 of the diaper, for attachment of the external connector.

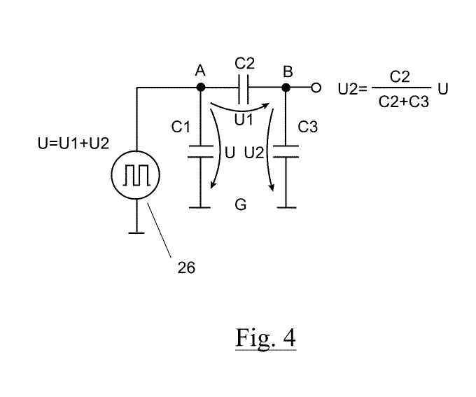

In the urination detection area 12 on the exterior side, opposite to the surface of diaper 22, the flexible PCB 16 has a conductive thin surface G connected to ground, as shown in FIG. 4. On the other side, the side which faces the surface of diaper 22, there are two thin conductive surfaces A and B which may be releasably attached to the surface of diaper 22 by a weak adhesive. Conductive surfaces A, B and G may be copper. Between them is a dielectric layer D as shown in FIG. 3. Some range of thicknesses is possible for D such as 0.1 mm to 3 mm, but this can be altered given the desired capacitance and di-electric constant of the material to achieve a desired capacitance. Most significantly, the dielectric layer D must be flexible and preferably retain no memory of shape after being bent. Surface A forms a fixed-value capacitor C1 with surface G. Surface B forms another fixed-value capacitor C3 with conductive surface G. Surfaces A and B then form a variable capacitor C2 having the diaper 22 material itself as dielectric. Diaper humidity content influences the value of this variable capacitor C2. Due to effects such as ion conduction through saline and other content of urine, additional resistive elements may be present in the general case electrical impedance of the sensor elements that are presented as capacitive in FIG. 4. However, capacitance-only analysis suffices for the detection purposes and for the AC measurement methods described as follows; more complex analysis could be performed, such as using multiple measurement frequencies, to extract general case resistive and reactive impedance component values, but in practice it is found that this is not necessary. The flexible PCB 16 and any attached components may also be coated with a coating resistant or otherwise impermeable to water and other chemicals that may be present in urine and fecal matter.

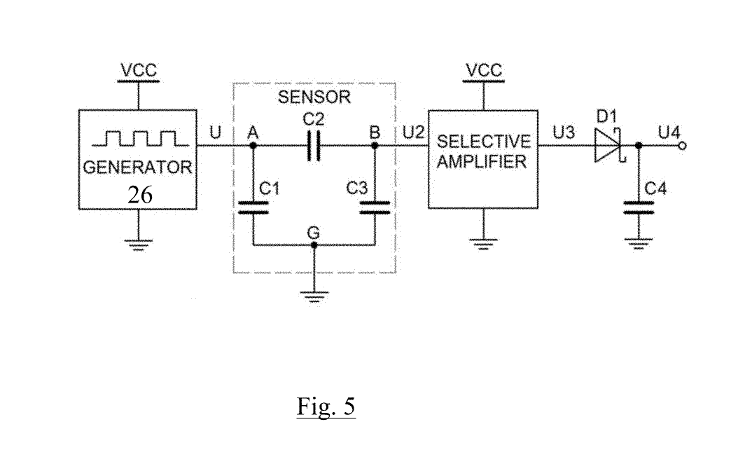

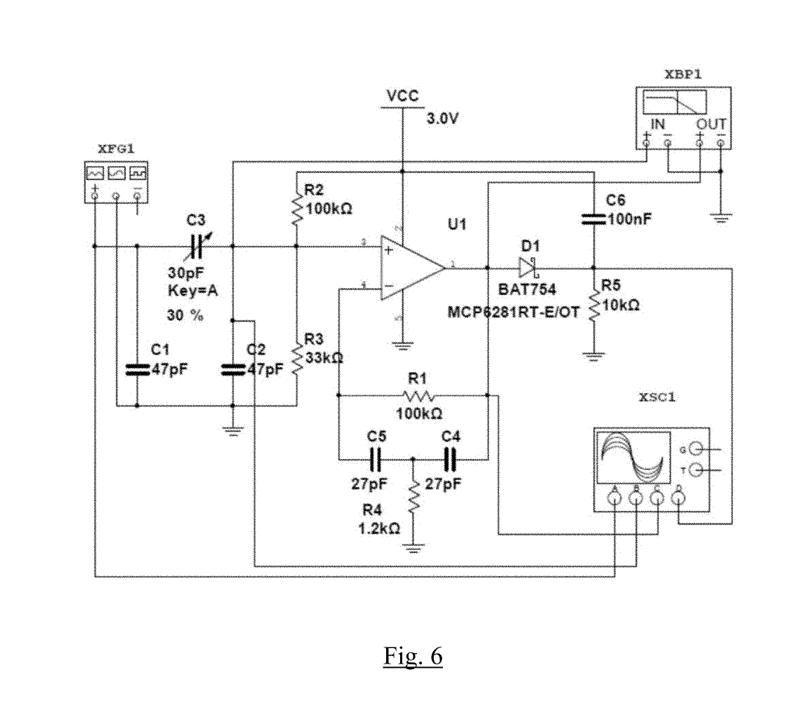

Sensor capacitances AG, AB and BG can be measured by a variety of electronic instrumentation means within the scope of the invention. Two such non-limiting examples are shown in FIGS. 5 and 6. In both FIGS. 5 and 6, C1 corresponds to capacitance across AG that as shown in FIGS. 1. C2 and C3 are the other two capacitances corresponding to AB and BG of FIG. 1.

FIG. 5 shows key elements of one representative capacitance measurement system appropriate for sensor 10. The square pulse signal of amplitude U generated at generator output 26 is applied to sensor 10, as shown in FIG. 4. Optimal frequency can be determined experimentally for maximum sensitivity and minimum body influence. Sensor output which is on C3 is a square wave signal having amplitude expressed by the following:

$$U 2 = \frac{C 2}{C 2 + C 3} U$$

in which U=constant applied signal, C3=constant, so U2 is proportional to the liquid quantity in diaper 22.

The following is a representative value example:

1. Dry diaper: U=2.8V, C2=2pF, C3=100pF, and U2=0.055V

2. Diaper full: U=2.8V, C2=20pF, C3=100pF, and U2=0.47V

U2 is applied to a frequency-selective amplifier (see FIG. 5) with sinuisoidal output U3.
U3 is rectified with diode D1 and filtered with C4. The resultant output voltage for detection and measurement purposes is voltage U4. The amplification factor is chosen in such way that with diaper 22 full, U3 is at or below the maximum permitted value at the analog-to-digital (A/D) processor input. In this way, the sensor and system can provide both relative indication of initial and increasing urinary output. In addition, an estimate for the total absolute quantity of urine to saturation level for the diaper or undergarment may be obtained from calibration of capacitance in empty and saturated states.

In FIG. 6 is shown another example of a supporting electronics instrumentation system for the invention. In this case a more detailed schematic representation of key electronic circuit components plus functional representations of standard electronic instruments is included, than in the functional representation of FIG. 5. The connected capacitors as shown are supplied with an AC signal via a signal generator/function generator XFG1. Oscilloscope XSC1 may be provided for viewing the signal as a function of time. XBP1 is a Bode Plotter for plotting signal amplitude as a function of frequency, i.e. frequency response. The resultant voltage is processed, amplified and introduced at the analog-to-digital (A/D) input of a microprocessor or other integrated circuit having such A/D capability (not shown). The output voltage value then varies generally proportionally with liquid quantity in the diaper 22. Having the surface G over A and B and connected to ground brings the advantage of reducing the influence of the patient's body in detection and measurement. This can be further optimised via selection of an appropriate measurement frequency for the function generator depending on the thickness of dielectric D and the equivalent surface of G. In general, some optimization of frequency is possible for purposes such as maximizing detection sensitivity and reducing effects of stray impedances from the patient's body and other sources. FIG. 6 labelling includes numeric values in standard units for passive components in the analytic circuitry, those components being resistors R1-R5 and capacitors C4, C5, and C6, to provide one reference embodiment as representative examples. However, alternate implementations of the circuitry may use somewhat different values and configurations for these components as will be apparent to one skilled in the art.

As noted previously, on the flexible PCB 16 of sensor apparatus 10, there are two areas with distinct purposes. One area 12 is for urination detection and measurement and the other area 14 is for defecation detection. For better event detection, on both urination and defecation areas 12, 14 there are a multitude of thermistors 20, 24 respectively, equally distributed over the length and surface of these areas. These thermistors 20, 24 are series connected for each of the two areas and necessary connections for these may be made, for example, by soldering to appropriately configured conductive areas on the sensor PCB surface 16, or by other means of electrical connection. The detective thermistor groups are then each connected to the output connector 18 of sensor 10. There is one thermistor output of the combined series resistance of the thermistors 20 of the urination area 12 for urination detection. There is similarly a second output resulted from thermistors 24 in defecation area 14, which are series connected. In the case as shown in FIG. 5, the thermistors' output signals may communicate to an A/D input of a detector microprocessor (not shown). Capacitive sensor ABG output may be transmitted to another detector microprocessor A/D input (not shown).

In one variant on the above-described sensor apparatus, there may also be another capacitive detection unit configured similar to the previously described AB and AG, but located on the opposite side of the sensor 10. This addition to the sensor 10 may increase the urination detection and measurement area for increased sensitivity.

There is therefore provided a sensing apparatus for the detection of urination events by persons or animals, employing a plurality of capacitive elements, each element comprised of one planar conductor opposite a conducting shielding layer common to all the capacitive elements, and for which the sensing apparatus is attached to undergarments or other attire to be worn by a human or animal patient. One or more thermistors may be connected in series are affixed to the sensing apparatus for the thermal detection of defecation events. Two independent thermistors or two sets of multiple thermistors connected in series may be affixed to the apparatus for the independent detection, respectively, of defecation and urination events. The sensing elements may be mounted on a flexible printed circuit board. Capacitance measuring techniques may be used with the apparatus for the monitoring of relative quantities of urinary output as a function of time. Capacitance measuring techniques may be used with the apparatus along with calibration of urinary output absorbed quantities, for the monitoring of absolute quantities of urinary output.

A system for monitoring urinary incontinence and remotely alerting caregivers of urination events is thereby provided which employs the aforementioned sensing apparatus for the purpose of detecting urination events as well as a system for incontinence monitoring, employing the aforementioned sensing apparatus for the purpose of separately detecting urination and defecation events.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

1. A sensing apparatus for use in the detection and assessment of urination events in a garment worn by a person, said sensing apparatus comprising a plurality of capacitive elements, each said capacitive element comprising a planar conductor having a first planar surface for releasable attachment to the outer surface of said garment and an opposed surface mounted on and dielectrically spaced from a grounded conductive shielding layer common to said plurality of capacitive elements, the planar conductor of each said capacitive element being isolatedly spaced from each adjacent planar conductor whereby each said planar conductor in combination with said grounded shielding layer forms a first plurality of capacitive elements and said outer surface of said garment in combination with a pair of adjacent planar conductors forms a second capacitive element in which a portion of said outer surface of said garment connecting said adjacent planar conductors forms the dielectric element of said second capacitive element.

2. The sensing apparatus of claim 1 wherein said plurality of capacitive elements comprises two spaced co-planar conductors each having a first planar surface for attachment to the outer surface of said garment and a second opposed surface attached to a first surface of a dielectric layer, a second surface of said dielectric layer being attached to said grounded conductive shielding layer.

3. The sensing apparatus of claim 1 further comprising a section for detection of defecation events.

4. The sensing apparatus of claim 3 further comprising one or more thermistors connected in series mounted on or within said sensing apparatus for the thermal detection of defecation events.

5. The sensing apparatus of claim 3 further comprising two independent thermistors or two independent pluralities of thermistors connected in series mounted on or within said sensing apparatus for the independent detection of defecation and urination events.

6. The sensing apparatus of claim 1 in which said plurality of capacitive elements are mounted on a flexible printed circuit board.

7. A system for using capacitance measuring techniques to monitor relative quantities of urinary output of a person wearing a garment as a function of time, said system comprising the sensing apparatus of claim 1, a function generator for generating an AC signal of selected frequency communicated to said sensing apparatus, and a selective amplifier for generating from an AC signal from said sensing apparatus a signal representing a variable voltage for processing in an analog-to-digital processor to provide a relative indication of urinary output in said garment.

8. A method of using capacitance measuring techniques with the system of claim 7, with calibration of urinary output absorbed quantities, for monitoring the quantities of urinary output in a person's garment.

9. The method of claim 8 wherein the quantity of urinary output in said garment is calculated using the equation:
$$U 2 = \frac{C 2}{C 2 + C 3} U$$
in which U=the AC signal of selected frequency, C3=a constant being the capacitance between said first planar conductor and said conductive shielding layer, and C2 is the variable capacitance between said two co-planar conductors, whereby U2 is proportional to the quantity of urinary output in said garment.

10. A system for monitoring urinary incontinence and remotely alerting caregivers of urination events, said system comprising the system of claim 7 for detecting urination events and the significance of such urinary events and means for communicating the detection of said events to said caregivers.

11. A system for incontinence monitoring and remotely alerting caregivers of incontinence events, comprising the system of claim 7 for detecting urination and defecation events and the significance of such urinary events and means for communicating the detection of said events to said caregivers.
